(19) United States
(12) Patent Application Publication
(10) Pub. No.: US 2019/0240024 A1
(43) Pub. Date: Aug. 8, 2019

(54) Tissue Anchors with Flexible Tips for Insertion into the Pericardial Cavity

(71) Applicant: 4Tech Inc., Waltham, MA (US)

(72) Inventors: Idan Tobis, Beth Hashmonai (IL); Jean Claude Laborde, Nottingham (GB); Gabriel Sobrino-Serrano, Kinvara (IE); Patrick Griffen, Castlegar (IE)

(73) Assignee: 4Tech Inc., Waltham, MA (US)

(21) Appl. No.: 16/326,332

(22) PCT Filed: Aug. 17, 2017
(86) PCT No.: PCT/US2017/047442

§ 371 Date: Feb. 18, 2019

Related U.S. Application Data
(63) Continuation-in-part of application No. PCT/IL2017/050771, filed on Jul. 10, 2017.
(60) Provisional application No. 62/376,685, filed on Aug. 18, 2016.

(51) Int. Cl. A61F 2/24

(52) U.S. Cl. CPC A61F 2/2478; A61F 2/2466; A61M 25/0054; A61F 2230/0091; A61F 2250/0009; A61F 2002/249

(57) ABSTRACT

An expandable tissue anchor (20, 120, 190, 220, 320, 420) includes an elongate tissue-coupling portion (30, 130, 192, 230, 330, 430) supported by an anchor shaft (32) at a first end (34) of the tissue-coupling portion (30, 130, 192, 230, 330, 430). The tissue-coupling portion (30, 130, 192, 230, 330, 430) is configured to be delivered in an unexpanded generally elongate configuration through a cardiac tissue wall from a first side of the wall to a second side of the wall, and to expand, on the second side of the cardiac tissue wall, to an expanded open shape configuration, such that the expanded tissue-coupling portion (30, 130, 192, 230, 330, 430) can be drawn tightly against the second side of the cardiac tissue wall when a tensile force is applied to the tissue-coupling portion (30, 130, 192, 230, 330, 430). The anchor (20, 120, 190, 220, 320, 420) also includes an elongate tip portion (38, 138, 238, 338, 438) supported at a second end (40, 240, 340, 440) of the tissue-coupling portion (30, 130, 192, 230, 330, 430) and configured to be delivered through the cardiac tissue wall ahead of the tissue-coupling portion (30, 130, 192, 230, 330, 430), the tip portion (38, 138, 238, 338, 438) including material having a stiffness less than a stiffness of the tissue-coupling portion (30, 130, 192, 230, 330, 430).

### CROSS-REFERENCE TO RELATED APPLICATIONS

The present application claims priority from (a) International Application PCT/IL2017/050771, filed Jul. 10, 2017, and (b) U.S. Provisional Application 62/376,685, filed Aug. 18, 2016, which are assigned to the assignee of the present application and are incorporated herein by reference.

### FIELD OF THE APPLICATION

The present invention relates generally to tissue anchors, and specifically to tissue anchors for implantation in soft tissue, such as cardiac tissue.

### BACKGROUND OF THE APPLICATION

Tissue anchors are used for anchoring elements, such as pacemaker electrode leads or sutures, to tissue, such as bone or soft tissue. PCT Publication WO 2016/087934 to Gilmore et al., which is incorporated in its entirety herein by reference, describes a tissue anchor that includes a shaft, a tissue-coupling element, and a flexible elongate tension member. The tissue-coupling element includes a wire, which is shaped as an open loop coil having, in some applications, more than one coil revolution when the tissue anchor is unconstrained, i.e., expanded from a linear state to a coiled state. The tension member includes a distal portion, that is fixed to a site on the open loop coil, a proximal portion, which has a longitudinal segment that runs alongside at least a portion of the shaft, and a crossing portion, which (i) is disposed between the distal and the proximal portions along the tension member, and (ii) crosses at least a portion of the open loop when the tissue anchor is expanded. The tissue anchor is configured to allow relative axial motion between the at least a portion of the shaft and the longitudinal segment of the proximal portion of the tension member when the tissue anchor is expanded.

US Patent Application Publication 2002/0013571 to Goldfarb et al. describes methods and devices for grasping, and optional repositioning and fixation of the valve leaflets to treat cardiac valve regurgitation, particularly mitral valve regurgitation.

### SUMMARY OF THE APPLICATION

Embodiments of the present invention provide an expandable tissue anchor that is deliverable to a cardiac chamber in an unexpanded generally elongate configuration within a deployment tool. The expandable tissue anchor is configured to be anchored to a cardiac tissue wall at a target site such that a tensile force can be applied to the expandable tissue anchor and thus to the cardiac tissue wall, once the expandable tissue anchor is deployed, so as to move the cardiac tissue wall at the target site relative to adjacent cardiac tissue. For some applications, such motion alters the geometry of a cardiac valve, such as the tricuspid valve or the mitral valve.

An expandable tissue anchor is provided configured to be deliverable to a cardiac chamber in an unexpanded generally elongate configuration within a percutaneous deployment tool, where the expandable tissue anchor is configured to be anchored to a cardiac tissue wall at a target site such that a tensile force can be applied to the expandable tissue anchor and thus to the cardiac tissue wall, once the expandable tissue anchor is deployed, so as to move the cardiac tissue wall at the target site relative to adjacent cardiac tissue. In some applications, the expandable tissue anchor comprises (1) an elongate tissue-coupling portion supported by an anchor shaft at a first end of the tissue-coupling portion, where the tissue-coupling portion is configured to be delivered in an unexpanded generally elongate configuration through the cardiac tissue wall from a first side of the wall to a second side of the wall, the tissue-coupling portion is further configured to expand, on the second side of the cardiac tissue wall, to an expanded open shape coil configuration, such that the expanded tissue-coupling portion can be drawn tightly against the second side of the cardiac tissue wall at the target site when the tensile force is applied to the tissue-coupling portion, and where the tissue-coupling portion comprising material having a first stiffness, and (2) an elongate tip portion supported at a second end of the tissue-coupling portion and configured to be delivered through the cardiac tissue wall ahead of the tissue-coupling portion, where the tip portion comprises material having a second stiffness less than the first stiffness.

In some applications, the tissue-coupling portion, once expanded on the second side of the cardiac tissue wall, comprises a configuration generally orthogonal to the anchor shaft. In some applications, the tip portion is configured such that when the cardiac tissue wall is a myocardial tissue wall, the tip portion can be directed, upon deployment in the pericardial cavity on the second side of the myocardial tissue wall, into the pericardial cavity ahead of the tissue-coupling portion, generally alongside and against pericardial tissue surrounding the myocardial tissue wall, without penetrating the pericardial tissue.

The tip portion may be axially fixed with respect to the tissue-coupling portion, or may be integral to the tissue-coupling portion. In some applications, the tip portion comprises a core wire and a coil wire wound around the core wire. In some applications, the tissue-coupling portion comprises a proximal portion and a distal bullet head, which is fixed to a distal end of the proximal portion, and has, at a widest longitudinal site along the bullet head, a greatest outer cross-sectional area that equals at least 150% of an average outer cross-sectional area of the proximal portion, and the bullet head serves as a crimp to secure the coil wire of the tip portion to the tissue-coupling portion.

In some applications, the tissue-coupling portion and the tip portion comprise a hollow tube. In some applications, the first stiffness of the tissue-coupling portion is at least 10% greater than the second stiffness of the tip portion. In some applications, the tip portion has a length of at least 3 mm. In some applications, the tip portion is not axially compressible.

In some applications, the tip portion comprises a plurality of slits that provides the lower stiffness of material than the tissue-coupling portion. In some cases, an average outer diameter of the tip portion is less than an average outer diameter of the tissue-coupling portion so as to provide the lesser lower stiffness of the material of the tip portion.

In some applications, the tip portion comprises a core wire and a coil wire wound around the core wire. In some applications, the tissue-coupling portion comprises a proximal portion and a distal bullet head, which is fixed to a distal end of the proximal portion, and has, at a widest longitudinal site along the bullet head, a greatest outer cross-sectional area that equals at least 150% of an average outer cross-sectional area of the proximal portion, and the bullet head serves as a crimp to secure the tip portion to the tissue-coupling portion.

In some applications, the expandable tissue anchor further comprises an elongate tension member coupled to a portion of the tissue-engaging coupling portion such that the tensile force can be applied to the tissue-coupling portion after it has been expanded to the open shape configuration. In some applications, the tip portion is an integral distal extension of the elongate tension member. In some applications, the tip portion is shaped as a closed loop, with a distal end of the elongate tension member fixed to the tissue-coupling portion near the second end of the tissue-coupling portion. In some applications, the tissue-coupling portion comprises a proximal portion and a distal bullet head, which is fixed to a distal end of the proximal portion, and has, at a widest longitudinal site along the bullet head, a greatest outer cross-sectional area that equals at least 150% of an average outer cross-sectional area of the proximal portion, and the bullet head serves as a crimp to secure the distal end of the elongate tension member to the tissue-coupling portion.

In some applications, an anchor system is provided that comprises the tension member expandable tissue anchor applications contemplated herein, and further comprises a tether affixed to the elongate tension member such that the tensile force can be applied to the expandable tissue anchor via the tether and the elongate tension member. In some applications, the anchor system comprises a second expandable tissue anchor, separate and distinct from the first expandable tissue anchor.

There is further provided, in accordance with an application of the present invention, a method for moving a cardiac tissue wall at a target site relative to adjacent cardiac tissue, the method including:

delivering, to a cardiac chamber, an expandable tissue anchor in an unexpanded generally elongate configuration within a deployment tool, the expandable tissue anchor including (a) an elongate tissue-coupling portion supported by an anchor shaft at a first end of the tissue-coupling portion, and including material having a first stiffness and (b) an elongate tip portion supported at a second end of the tissue-coupling portion, and including material having a second stiffness less than the first stiffness;

delivering the elongate tip portion through the cardiac tissue wall ahead of the tissue-coupling portion;

delivering the tissue-coupling portion in an unexpanded generally elongate configuration through the cardiac tissue wall from a first side of the wall to a second side of the wall, such that the tissue-coupling portion expands, on the second side of the cardiac tissue wall, to an expanded open shape configuration, thereby anchoring the expandable tissue anchor to the cardiac tissue wall at the target site; and

tightly drawing the expanded tissue-coupling portion against the second side of the cardiac tissue wall at the target site by applying a tensile force to the tissue-coupling portion, and thus to the cardiac tissue wall, so as to move the cardiac tissue wall at the target site relative to the adjacent cardiac tissue.

For some applications, the cardiac tissue wall is a myocardial tissue wall, and delivering the tip portion through the cardiac tissue wall ahead of the tissue-coupling portion includes directing the tip portion, upon deployment in the pericardial cavity on the second side of the myocardial tissue wall, into the pericardial cavity ahead of the tissue-coupling portion, generally alongside and against pericardial tissue surrounding the myocardial tissue wall, without penetrating the pericardial tissue.

For some applications, applying a tensile force to the tissue-coupling portion includes applying the tensile force to an elongate tension member coupled to a portion of the tissue-coupling portion. For some applications, applying the tensile force to the elongate tension member includes applying the tensile force to a tether affixed to the elongate tension member.

For some applications, the method further includes implanting a second expandable tissue anchor, separate and distinct from the expandable tissue anchor.

The present invention will be more fully understood from the following detailed description of embodiments thereof, taken together with the drawings, in which:

### BRIEF DESCRIPTION OF THE DRAWINGS

FIG. 1 is a schematic illustration of an expandable tissue anchor, in accordance with an application of the present invention;

FIGS. 2A-B are schematic illustrations of additional configurations of another expandable tissue anchor, in accordance with respective applications of the present invention;

FIG. 3 is a schematic illustration of yet another configuration of yet another expandable tissue anchor, in accordance with an application of the present invention;

FIG. 4 is a schematic illustration of still another configuration of still another expandable tissue anchor, in accordance with an application of the present invention;

FIG. 5 is a schematic illustration of an additional configuration of additional expandable tissue anchor, in accordance with an application of the present invention; and

FIGS. 6A-D are schematic illustrations of method of deploying the expandable tissue anchor of FIG. 1 through a myocardial tissue wall, in accordance with an application of the present invention.

### DETAILED DESCRIPTION OF APPLICATIONS

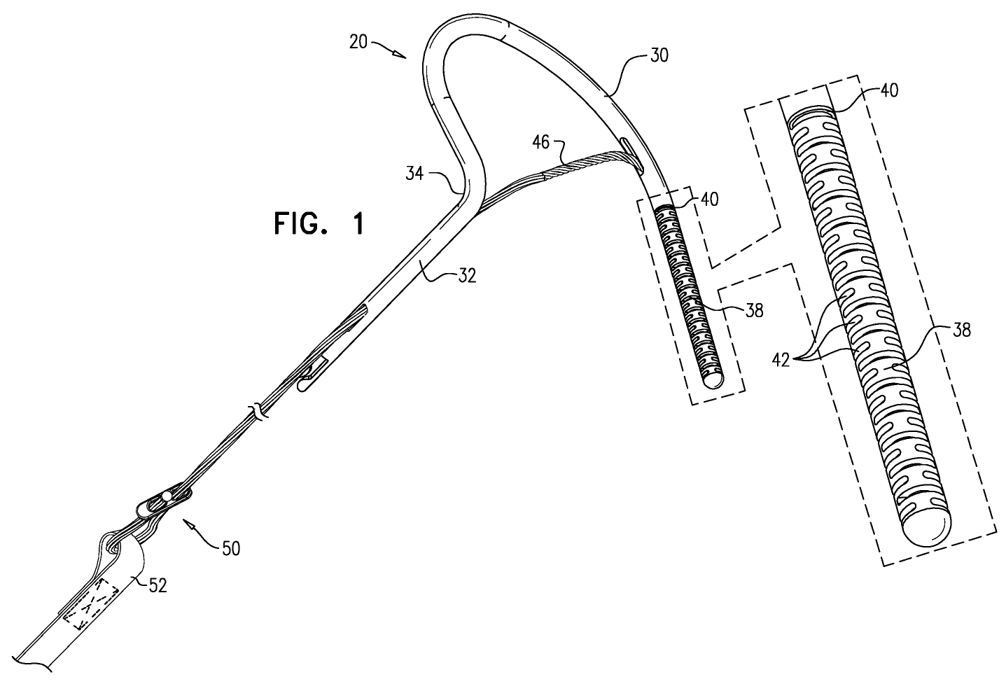

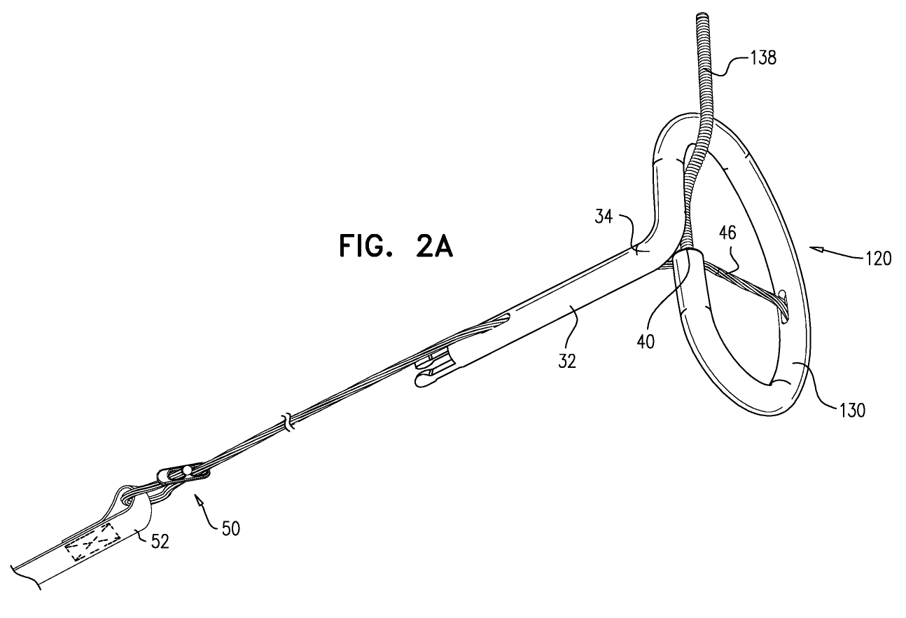

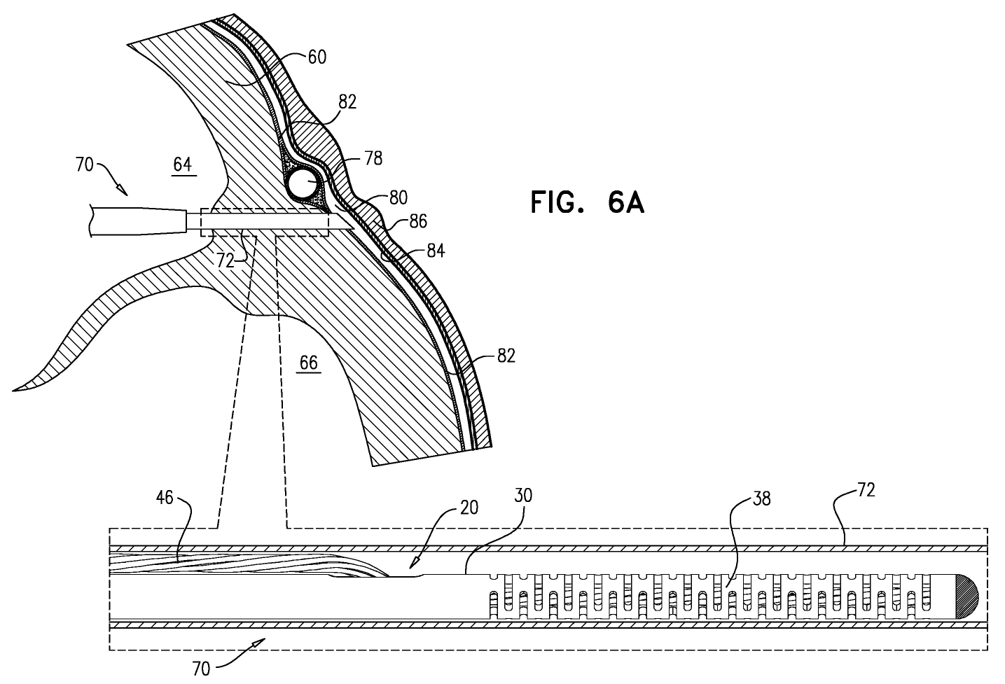

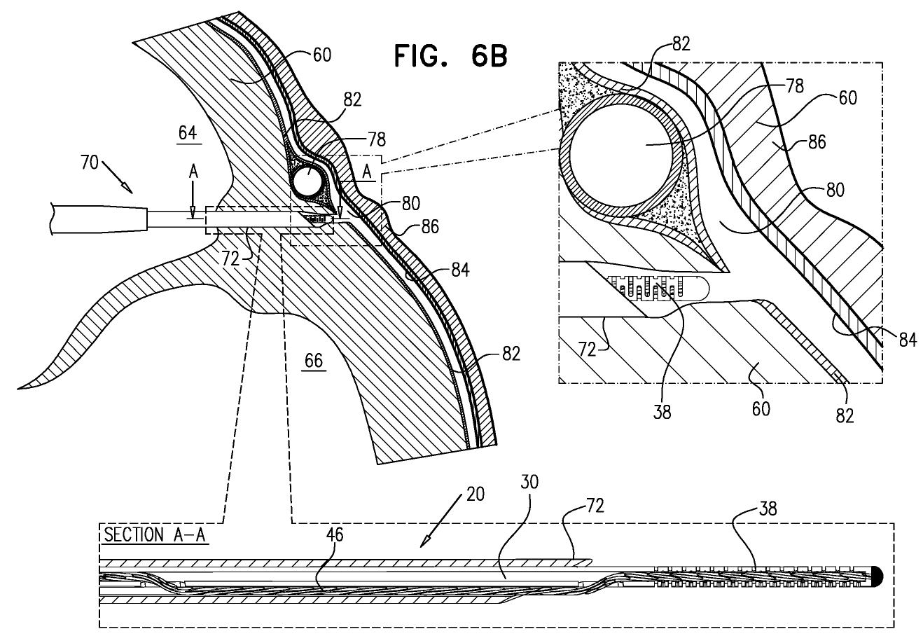

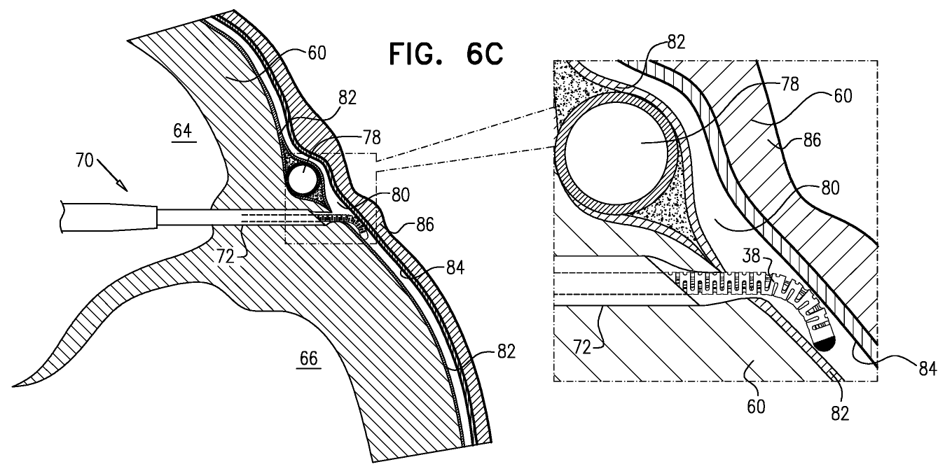

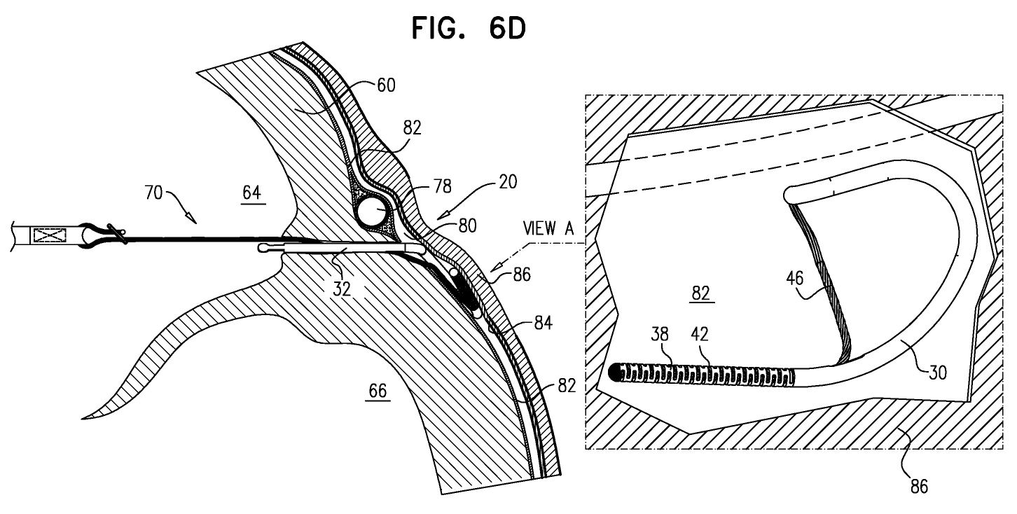

Referring to FIG. 1, an expandable tissue anchor 20 is provided that is configured to be anchored to a cardiac tissue wall at a target site such that a tensile force can be applied to expandable tissue anchor 20 and thus to the cardiac tissue wall, so as to move the cardiac tissue wall at the target site relative to adjacent cardiac tissue. Expandable tissue anchor 20 comprises an elongate tissue-coupling portion 30 supported by an anchor shaft 32 at a first end 34 of tissue-coupling portion 30. Tissue-coupling portion 30 is configured to be delivered in an unexpanded generally elongate configuration, such as described hereinbelow with reference to FIG. 6A, through the cardiac tissue wall from a first side of the wall to a second side of the wall, such as described hereinbelow with reference to FIGS. 6A-D. Tissue-coupling portion 30 is further configured, upon deployment, to expand, on the second side of the cardiac tissue wall, to an expanded coil (open shape) configuration, such as described hereinbelow with reference to FIG. 6D, such that the expanded tissue-coupling portion 30 can be drawn tightly against the second side of the cardiac tissue wall at the target site when the tensile force is applied to tissue-coupling portion 30. Tissue-coupling portion 30 comprises material having a first stiffness.

Expandable tissue anchor 20 further comprises an elongate tip portion 38 supported at a second end 40 of tissue-coupling portion 30 and configured to be delivered through the cardiac tissue wall ahead of tissue-coupling portion 30, such as described hereinbelow with reference to FIGS. 6A-D. Tip portion 38 comprises material having a second stiffness less than the first stiffness of the material of tissue-coupling portion 30. For example, the first stiffness of tissue-coupling portion 30 may be at least 10% greater than the second stiffness of tip portion 38, such as 20% or 30% greater.

For some applications, tissue-coupling portion 30, once expanded on the second side of the cardiac tissue wall, such as described hereinbelow with reference to FIG. 6D, comprises a configuration generally orthogonal to anchor shaft 32, although it need not be orthogonal. For some applications, such as shown in FIG. 1, tip portion 38 is comprises a plurality of slits 42 that provides the lower stiffness of material of tip portion 38 than of tissue-coupling portion 30. For some applications, the tissue-coupling portion 30 and tip portion 38 comprise the same material, but they need not. Slits 42 may be oriented circumferentially around tip portion 38 (as shown); axially along tip portion 38 (configuration not shown); in another orientation, such as helically around tip portion 38 (configuration not shown); or in a plurality of different orientations. For some applications, such as shown in FIG. 1, tissue-coupling portion 30 and tip portion 38 are integral to one another, although they may also be discrete components fixed together. For some applications, tissue-coupling portion 30 and tip portion 38 comprise a hollow tube. In some applications, tip portion 38 is not axially compressible. For some applications, tip portion 38 has a length of at least 3 mm, such as at least 5 mm, or even at least 20 mm (but typically no more than 60 mm), which may additionally provide a clear indication of pericardial placement.

Referring to FIG. 1, for some applications, expandable tissue anchor 20 further comprises an elongate tension member 46 coupled to a portion of tissue-coupling portion 30. Through elongate tension member 46, or components equivalent thereto, a tensile force can be applied to tissue-coupling portion 30 after it has been expanded to the coil open shape configuration. When applied in vivo, the tensile force may have the benefit of bringing the anchor close to the tissue wall to which it is applied. For some applications, an anchor system 50 is provided that comprises expandable tissue anchor 20 and a tether 52 affixed to elongate tension member 46 such that the tensile force can be applied to expandable tissue anchor 20 via tether 52 and elongate tension member 46. For some applications, anchor system 50 further comprises a second expandable tissue anchor, separate and distinct from expandable tissue anchor 20, such as is shown in PCT Publication WO 2016/087934 incorporated herein by reference. For some applications, the second expandable tissue anchor, and additional anchors if so desired, is couplable or coupled to expandable tissue anchor 20 by one or more tethers that include tether 52.

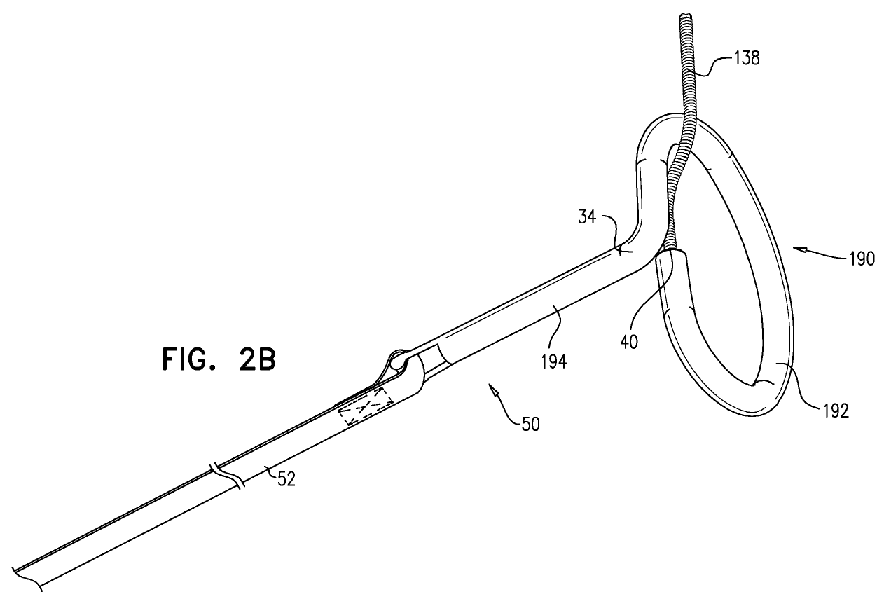

Referring to FIG. 2A, an expandable tissue anchor 120 is provided that is generally similar to expandable tissue anchor 20, except as described below. An elongate tip portion 138 of tissue anchor 120 may be made of the same or similar material as a tissue-coupling portion 130 of the tissue anchor, but the average outer diameter of tip portion 138 is less than an average outer diameter of tissue-coupling portion 130 so as to provide the lower stiffness of the material of tip portion 138 than of tissue-coupling portion 130. The difference in stiffness properties may be implemented by a difference in material and/or a difference in dimensions. For example, the average outer diameter of tip portion 138 may be at least 10% less, such as at least 20% less, than the average outer diameter of tissue-coupling portion 130.

Reference is still made to FIG. 2A, and is additionally made to FIG. 2B, which is a schematic illustration of an expandable tissue anchor 190 that is generally similar to expandable tissue anchor 120, except as described below. Like tissue anchor 120, tissue anchor 190 comprises a tissue-coupling portion 192, elongate tip portion 138, and an anchor shaft 194 at first end 34 of tissue-coupling portion 192. However, unlike in the configuration of tissue anchor 120 shown in FIG. 2A, tissue anchor 190 does not comprise elongate tension member 46. Tension is instead applied to tissue-coupling portion 192 through anchor shaft 194, typically via tether 52. Although, for the sake of brevity, configurations of tissue anchors 20, 220, 320, and 420 are not illustrated without elongate tension member 46, it is to be understood that elongate tension member 46 is optional in all of the tissue anchors described herein, and that all of the tissue anchors described herein may be provided without elongate tension member 46, as shown in FIG. 2B for tissue anchor 190, mutatis mutandis.

Referring to FIGS. 2A and 2B, for some applications, tissue-coupling portion 130 or 192 comprises a hollow tube, while for other applications, tissue-coupling portion 130 or 192 comprises a solid wire.

Referring to FIG. 3, an expandable tissue anchor 220 is provided that is generally similar to expandable tissue anchor 20, except as described below. An elongate tip portion 238 of tissue anchor 220 is provided as an integral distal extension of an elongate tension member 246, similar to elongate tension member 46 described hereinabove with reference to FIG. 1. In other words, a single elongate member (e.g., a solid wire or a hollow tube) defines both elongate tension member 246 and elongate tip portion 238. Elongate tip portion 238 is supported at, and extends distally beyond, a second end 240 of an elongate tissue-coupling portion 230. For some applications, as shown, elongate tip portion 238 is shaped as a closed loop 244, with a distal end 248 of elongate tension member 246 fixed to elongate tissue-coupling portion 230 near a second end 240 of the tissue-coupling portion. Typically, the average outer diameter of tip portion 238 is less than an average outer diameter of tissue-coupling portion 230 (e.g., at least 10% less, such as at least 20% less), so as to provide at least a portion of the lower stiffness of the material of tip portion 238 than of tissue-coupling portion 230.

In some configurations, elongate tissue-coupling portion 230 comprises a narrow proximal portion 236 and a distal bullet head 222 (shown in cross section in FIG. 3), which is fixed to a distal end of proximal portion 236, and has, at a widest longitudinal site along bullet head 222, a greatest outer cross-sectional area that equals at least 150% (e.g., at least 200%, or at least 300%) of an average outer cross-sectional area of proximal portion 236. (The cross-sectional area of bullet head 222 is measured perpendicular to a central longitudinal axis of the bullet head. Similarly, the cross-sectional area of proximal portion 236 is measured perpendicular to a central longitudinal axis of proximal portion 236.) Typically, a distal end of distal bullet head 222 defines second end 240 of elongate tissue-coupling portion 230.

Bullet head 222 may increase the surface area of tissue-coupling portion 230 for pressing against myocardial tissue wall 60, described hereinbelow with reference to FIGS. 6A-D, which may increase stability when force is applied to tissue-coupling portion 230. Bullet head 222 may also serve as a crimp to secure distal end 248 of elongate tension member 246 to elongate tissue-coupling portion 230. Typically, a portion of elongate tension member 246 passes through a channel defined through bullet head 222. Alternatively, the greatest outer cross-sectional area of bullet head 222 equals less than 150% of the average outer cross-sectional area of proximal portion 236, in which configuration bullet head 222 may serve only as a crimp for securing distal end 248 of elongate tension member 246 to elongate tissue-coupling portion 230.

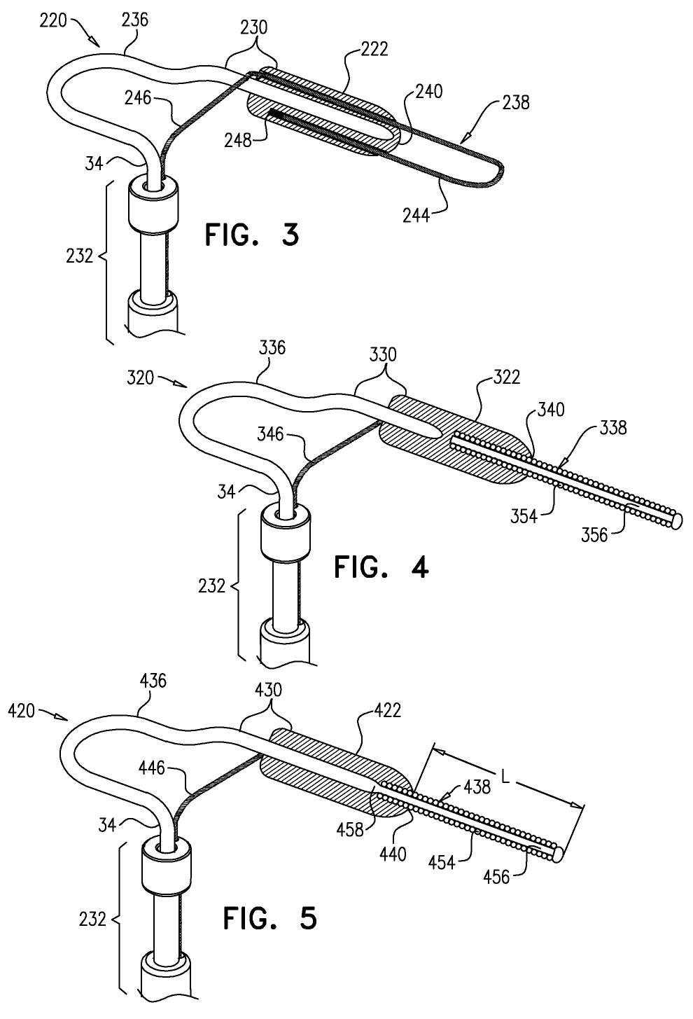

Referring to FIG. 4, an expandable tissue anchor 320 is provided that is generally similar to expandable tissue anchor 120, except as described below. An elongate tip portion 338 of tissue anchor 320 is supported at a second end 340 of an elongate tissue-coupling portion 330 of tissue anchor 320, and may be made of the same or similar material as tissue-coupling portion 330, but the average outer diameter of a core wire 356 of elongate tip portion 338 is typically less than an average outer diameter of tissue-coupling portion 330 so as to provide the lower stiffness of the material of tip portion 338 than of tissue-coupling portion 330. The difference in stiffness properties may be implemented by a difference in material and/or a difference in dimensions. For example, the average outer diameter of tip portion 338 may be at least 10% less, such as at least 20% less, than the average outer diameter of tissue-coupling portion 330.

For some applications, tip portion 338 comprises core wire 356 and a coil wire 354 wound around core wire 356. Core wire 356 may, for example, comprise Nitinol, and coil wire 354 may, for example, comprise a platinum-iridium alloy, and is typically radiopaque, such as for visualization under fluoroscopy.

In some configurations, elongate tissue-coupling portion 330 comprises a narrow proximal portion 336 and a distal bullet head 322 (shown in cross section in FIG. 4), which is fixed to a distal end of proximal portion 336, and has, at a widest longitudinal site along bullet head 322, a greatest outer cross-sectional area that equals at least 150% (e.g., at least 200%, or at least 300%) of an average outer cross-sectional area of proximal portion 336. (The cross-sectional area of bullet head 322 is measured perpendicular to a central longitudinal axis of the bullet head. Similarly, the cross-sectional area of proximal portion 336 is measured perpendicular to a central longitudinal axis of proximal portion 336.) A distal end of distal bullet head 322 defines second end 340 of elongate tissue-coupling portion 330.

Bullet head 322 may increase the surface area of tissue-coupling portion 330 for pressing against myocardial tissue wall 60, described hereinbelow with reference to FIGS. 6A-D, which may increase stability when force is applied to tissue-coupling portion 330. Bullet head 322 also serves as a crimp to secure tip portion 338 to elongate tissue-coupling portion 330. Alternatively, the greatest outer cross-sectional area of bullet head 322 equals less than 150% of the average outer cross-sectional area of proximal portion 336, in which configuration bullet head 322 may serve only as a crimp for securing tip portion 338 to elongate tissue-coupling portion 330.

Optionally, tissue anchor 320 further comprises an elongate tension member 346, similar to elongate tension member 46 described hereinabove with reference to FIG. 1.

Referring to FIG. 5, an expandable tissue anchor 420 is provided that is generally similar to expandable tissue anchor 320, except as described below. An elongate tip portion 438 of tissue anchor 420 is supported at a second end 440 of an elongate tissue-coupling portion 430 of tissue anchor 420, and may be made of the same or similar material as tissue-coupling portion 430, but the average outer diameter of a core wire 456 of elongate tip portion 438 is typically less than an average outer diameter of tissue-coupling portion 430 so as to provide the lower stiffness of the material of tip portion 438 than of tissue-coupling portion 430. The difference in stiffness properties may be implemented by a difference in material and/or a difference in dimensions. For example, the average outer diameter of tip portion 438 may be at least 10% less, such as at least 20% less, than the average outer diameter of tissue-coupling portion 430.

For some applications, elongate tissue-coupling portion 430 comprises a narrow proximal portion 436, and tip portion 438 comprises core wire 456 that is an integral distal extension of proximal portion 436 of tissue-coupling portion 430. Proximal portion 436 narrows at a tapering portion 458 to provide narrower core wire 456, as described above.

For some applications, tip portion 438 further comprises a coil wire 454 wound around core wire 456. Core wire 456 may, for example, comprise Nitinol, and coil wire 454 may, for example, comprise a platinum-iridium alloy, and is typically radiopaque.

In some configurations, elongate tissue-coupling portion 430 further comprises a distal bullet head 422 (shown in cross section in FIG. 5), which is fixed to a distal end of proximal portion 436, and has, at a widest longitudinal site along bullet head 422, a greatest outer cross-sectional area that equals at least 150% (e.g., at least 200%, or at least 300%) of an average outer cross-sectional area of proximal portion 436. (The cross-sectional area of bullet head 422 is measured perpendicular to a central longitudinal axis of the bullet head. Similarly, the cross-sectional area of proximal portion 436 is measured perpendicular to a central longitudinal axis of proximal portion 436.) A distal end of distal bullet head 422 defines second end 440 of elongate tissue-coupling portion 430.

Bullet head 422 may increase the surface area of tissue-coupling portion 430 for pressing against myocardial tissue wall 60, described hereinbelow with reference to FIGS. 6A-D, which may increase stability when force is applied to tissue-coupling portion 430. Bullet head 422 may also serve as a crimp to secure coil wire 454 of tip portion 438 to elongate tissue-coupling portion 430. Alternatively, the greatest outer cross-sectional area of bullet head 422 equals less than 150% of the average outer cross-sectional area of wire 436, in which configuration bullet head 422 may serve only as a crimp for securing coil wire 454 to elongate tissue-coupling portion 430.

Optionally, tissue anchor 420 further comprises an elongate tension member 446, similar to elongate tension member 46 described hereinabove with reference to FIG. 1.

For some applications, tip portion 438 has a length L, distally beyond second end 440 of tissue-coupling portion 430, of at least 3 mm, such as at least 5 mm, or even at least 20 mm (but typically no more than 60 mm), which may additionally provide a clear indication of pericardial placement. (Tip portion 438 of tissue anchor 320, described hereinabove with reference to FIG. 4, may also have this length.)

Referring to FIGS. 3-5, for some applications, narrow proximal portion 236, 336, or 436 comprises a solid wire, while for other applications, narrow proximal portion 236, 336, or 436 comprises a hollow tube.

Still referring to FIGS. 3-5, for some applications, elongate tip portion 238, 338, or 438 is configured to at least partially assume a spiral shape when unconstrained in the pericardial cavity 80, such as described hereinbelow with reference to FIGS. 6A-D.

Reference is now made to FIGS. 6A-D, which are schematic illustrations of a method of deploying expandable tissue anchor 20 through a myocardial tissue wall 60, in accordance with an application of the present invention. Although in FIGS. 6A-D expandable tissue anchor 20 is shown deployed through a myocardial tissue wall, expandable tissue anchor 20 may also be deployed through other cardiac tissue walls, such as the interatrial septum, either at or not at the fossa ovalis, or through other non-cardiac tissue walls. Indeed, the tissue anchors described herein may be deployed in any number of bodily locations where it is desired to anchor into or behind tissue for purposes of moving such tissue relative to adjacent tissue. The method of FIGS. 6A-D may also be used for deploying expandable tissue anchor 120, tissue anchor 190, tissue anchor 220, tissue anchor 320, and tissue anchor 420, described hereinabove with reference to FIGS. 2A, 2B, 3, 4, and 5, respectively.

As shown in FIG. 6A, expandable tissue anchor 20 is delivered to a target site, such as a cardiac chamber, in an unexpanded generally elongate configuration within a deployment tool 70, which comprises a hollow needle 72. The cardiac chamber may be a right atrium 64 (as shown), a right ventricle 66 (configuration not shown), a left atrium (configuration not shown), or a left ventricle (configuration not shown). In one application, hollow needle 72 is used to puncture through a first side of a myocardial tissue wall 60 and visceral pericardium 82 (which is part of the epicardium), avoiding vasculature such as the right coronary artery (RCA) 78. The deployment tool 70 is then further directed into the pericardial cavity 80 between visceral pericardium 82 and parietal pericardium 84, carefully avoiding puncturing parietal pericardium 84 and fibrous pericardium 86.

As shown in FIG. 6B, for some applications, hollow needle 72 is withdrawn slightly, exposing some or all of the tip portion of the expandable tissue anchor 20 within a bore created through myocardial tissue wall 60 by the hollow needle 72. As shown in FIG. 6C, elongate tip portion 38 of expandable tissue anchor 20 is delivered through myocardial tissue wall 60 ahead of tissue-coupling portion 30. Tip portion 38 can be directed, upon deployment in pericardial cavity 80 on the second side of myocardial tissue wall 60, into pericardial cavity 80 ahead of tissue-coupling portion 30, generally alongside and against pericardial tissue surrounding the myocardial tissue wall, without penetrating the pericardial tissue (particularly, parietal pericardium 84 and fibrous pericardium 86). Tip portion 38 is not stiff enough to inadvertently puncture the pericardial tissue.

As shown in FIG. 6D, tissue-coupling portion 30 of expandable tissue anchor 20 is delivered through myocardial tissue wall 60 and into pericardial cavity 80 following elongate tip portion 38. Elongate tip portion 38 directs tissue-coupling portion 30 generally alongside and against the pericardial tissue, thereby preventing the stiffer tissue-coupling portion 30 from puncturing the pericardial tissue (particularly, parietal pericardium 84 and fibrous pericardium 86). Tissue-coupling portion 30 expands to the coil open shape configuration on the second site of myocardial tissue wall 60, thereby anchoring expandable tissue anchor 20 to myocardial tissue wall 60.

Once expandable tissue anchor 20 has been anchored to myocardial tissue wall 60 at the target site, a tensile force is applied using tether 52 to expandable tissue anchor 20 and thus to myocardial tissue wall 60, so as to move myocardial tissue wall 60 at the target site relative to adjacent cardiac tissue. For some applications, such motion can have the benefit of altering the geometry of a nearby cardiac valve, such as the tricuspid valve or the mitral valve.

The scope of the present invention includes embodiments described in the following applications, which are assigned to the assignee of the present application and are incorporated herein by reference. For some applications, techniques and apparatus described in one or more of the following applications are combined with techniques and apparatus described herein: U.S. Pat. No. 8,475,525 to Maisano et al.; U.S. Pat. No. 8,961,596 to Maisano et al.; U.S. Pat. No. 8,961,594 to Maisano et al.; PCT Publication WO 2011/089601; U.S. Pat. No. 9,241,702 to Maisano et al.; PCT Publication WO 2013/011502; U.S. Provisional Application 61/750,427, filed Jan. 9, 2013; U.S. Provisional Application 61/783,224, filed Mar. 14, 2013; PCT Publication WO 2013/179295; U.S. Provisional Application 61/897,491, filed Oct. 30, 2013; U.S. Provisional Application 61/897,509, filed Oct. 30, 2013; U.S. Pat. No. 9,307,980 to Gilmore et al.; PCT Publication WO 2014/108903; PCT Publication WO 2014/141239; U.S. Provisional Application 62/014,397, filed Jun. 19, 2014; PCT Publication WO 2015/063580; US Patent Application Publication 2015/0119936; U.S. Provisional Application 62/086,269, filed Dec. 2, 2014; U.S. Provisional Application 62/131,636, filed Mar. 11, 2015; U.S. Provisional Application 62/167,660, filed May 28, 2015; PCT Publication WO 2015/193728; PCT Publication WO 2016/087934; US Patent Application Publication 2016/0242762; PCT Publication WO 2016/189391; and US Patent Application Publication 2016/0262741.

Patents and patent application publications incorporated by reference in the present patent application are to be considered an integral part of the application except that to the extent any terms are defined in these incorporated patents and patent application publications in a manner that conflicts with the definitions made explicitly or implicitly in the present specification, only the definitions in the present specification should be considered.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An expandable tissue anchor configured to be delivered to a cardiac chamber in an unexpanded generally elongate configuration within a deployment tool, the expandable tissue anchor configured to be anchored to a cardiac tissue wall at a target site such that a tensile force can be applied to the expandable tissue anchor and thus to the cardiac tissue wall, once the expandable tissue anchor is deployed, so as to move the cardiac tissue wall at the target site relative to adjacent cardiac tissue, the expandable tissue anchor comprising:
an elongate tissue-coupling portion supported by an anchor shaft at a first end of the tissue-coupling portion, the tissue-coupling portion configured to be delivered in an unexpanded generally elongate configuration through the cardiac tissue wall from a first side of the wall to a second side of the wall, the tissue-coupling portion further configured to expand, on the second side of the cardiac tissue wall, to an expanded configuration, such that the expanded tissue-coupling portion can be drawn tightly against the second side of the cardiac tissue wall at the target site when the tensile force is applied to the tissue-coupling portion, the tissue-coupling portion having a first stiffness; and
an elongate tip portion supported at a second end of the tissue-coupling portion and configured to be delivered through the cardiac tissue wall ahead of the tissue-coupling portion, the tip portion having a second stiffness less than the first stiffness.

2-7. (canceled)

8. The expandable tissue anchor according to claim 1, wherein the tip portion comprises a plurality of slits that provides the lower second stiffness of the tip portion.

9. The expandable tissue anchor according to claim 1, wherein an average outer diameter of the tip portion is less than an average outer diameter of the tissue-coupling portion so as to provide the lower second stiffness of the tip portion.

10. The expandable tissue anchor according to claim 1, wherein the tissue-coupling portion and the tip portion are integral to one another.

11-12. (canceled)

13. The expandable tissue anchor according to claim 10, wherein the tissue-coupling portion and the tip portion comprise a hollow tube.

14. The expandable tissue anchor according to claim 1, wherein the tip portion comprises a core wire and a coil wire wound around the core wire.

15. (canceled)

16. The expandable tissue anchor according to claim 1, further comprising an elongate tension member coupled to a portion of the tissue-coupling portion such that the tensile force can be applied to the tissue-coupling portion after it has been expanded to the expanded configuration.

17. The expandable tissue anchor according to claim 16,
wherein the tip portion is an integral distal extension of the elongate tension member, and
wherein the tip portion is shaped as a closed loop, with a distal end of the elongate tension member fixed to the tissue-coupling portion near the second end of the tissue-coupling portion.

18-19. (canceled)

20. An anchor system comprising the expandable tissue anchor according to claim 16, wherein the anchor system further comprises a tether affixed to the elongate tension member such that the tensile force can be applied to the expandable tissue anchor via the tether and the elongate tension member.

21-42. (canceled)

43. The expandable tissue anchor according to claim 1, wherein the tissue-coupling portion, once expanded on the second side of the cardiac tissue wall, comprises a configuration generally orthogonal to the anchor shaft.

44. The expandable tissue anchor according to claim 1, wherein the tip portion comprises a material different from that of the tissue-coupling portion so as to provide the lower second stiffness of the tip portion relative to the first stiffness of the tissue-coupling portion.

45. The expandable tissue anchor according to claim 1, wherein the tissue-coupling portion comprises a proximal portion and a distal bullet head, which is fixed to a distal end of the proximal portion, and has, at a widest longitudinal site along the bullet head, a greatest outer cross-sectional area that equals at least 150% of an average outer cross-sectional area of the proximal portion.

46. A method for moving a cardiac tissue wall at a target site relative to adjacent cardiac tissue, the method comprising:
delivering, to a cardiac chamber, an expandable tissue anchor in an unexpanded generally elongate configuration within a deployment tool, the expandable tissue anchor comprising (a) an elongate tissue-coupling portion supported by an anchor shaft at a first end of the tissue-coupling portion, and having a first stiffness and (b) an elongate tip portion supported at a second end of the tissue-coupling portion, and having a second stiffness less than the first stiffness;
delivering the elongate tip portion through the cardiac tissue wall ahead of the tissue-coupling portion;
delivering the tissue-coupling portion in an unexpanded generally elongate configuration through the cardiac tissue wall from a first side of the wall to a second side of the wall, such that the tissue-coupling portion expands, on the second side of the cardiac tissue wall, to an expanded configuration, thereby anchoring the expandable tissue anchor to the cardiac tissue wall at the target site; and
tightly drawing the expanded tissue-coupling portion against the second side of the cardiac tissue wall at the target site by applying a tensile force to the tissue-coupling portion, and thus to the cardiac tissue wall, so as to move the cardiac tissue wall at the target site relative to the adjacent cardiac tissue.

47. The method according to claim 46, wherein the tissue-coupling portion, once expanded on the second side of the cardiac tissue wall, comprises a configuration generally orthogonal to the anchor shaft.

48. The method according to claim 46, wherein the cardiac tissue wall is a myocardial tissue wall, and wherein delivering the tip portion through the cardiac tissue wall ahead of the tissue-coupling portion comprises directing the tip portion, upon deployment in the pericardial cavity on the second side of the myocardial tissue wall, into the pericardial cavity ahead of the tissue-coupling portion, generally alongside and against pericardial tissue surrounding the myocardial tissue wall, without penetrating the pericardial tissue.

49. The method according to claim 46, wherein the tip portion comprises a plurality of slits to provide the lower second stiffness of the tip portion.

50. The method according to claim 46, wherein an average outer diameter of the tip portion is less than an average outer diameter of the tissue-coupling portion so as to provide the lower second stiffness of the tip portion.

51. The method according to claim 46, wherein the tissue-coupling portion and the tip portion are integral to one another.

52. The method according to claim 51, wherein the tissue-coupling portion and the tip portion comprise a hollow tube.

53. The method according to claim 46, wherein the tip portion comprises a core wire and a coil wire wound around the core wire.

54. The method according to claim 46, wherein applying a tensile force to the tissue-coupling portion comprises applying the tensile force to an elongate tension member coupled to a portion of the tissue-coupling portion.

55. The method according to claim 54,
wherein the tip portion is an integral distal extension of the elongate tension member, and
wherein the tip portion is shaped as a closed loop, with a distal end of the elongate tension member fixed to the tissue-coupling portion near the second end of the tissue-coupling portion.

56. The method according to claim 54, wherein applying the tensile force to the elongate tension member comprises applying the tensile force to a tether affixed to the elongate tension member.

57. The method according to claim 46, wherein the tip portion comprises a material different from that of the tissue-coupling portion so as to provide the lower second stiffness of the tip portion relative to the first stiffness of the tissue-coupling portion.

58. The method according to claim 46, wherein the tissue-coupling portion comprises a proximal portion and a distal bullet head, which is fixed to a distal end of the proximal portion, and has, at a widest longitudinal site along the bullet head, a greatest outer cross-sectional area that equals at least 150% of an average outer cross-sectional area of the proximal portion.
